(19) United States
(12) Patent Application Publication
(10) Pub. No.: US 2021/0121133 A1
(43) Pub. Date: Apr. 29, 2021

(54) SYSTEM AND METHOD FOR RISK DETECTION AND INTERVENTION TO PREVENT SUDDEN DEATH

(71) Applicant: Christiana Care Health Services, Inc., Newark, DE (US)

(72) Inventors: John Pollard, Wallingford, PA (US); Chalita Atallah, Abingdon, MD (US)

(21) Appl. No.: 17/083,412

(22) Filed: Oct. 29, 2020

Related U.S. Application Data
(60) Provisional application No. 62/927,297, filed on Oct. 29, 2019.

(51) Int. Cl. A61B 5/00; A61B 5/11; A61B 5/0205; A61B 5/145

(52) U.S. Cl. CPC A61B 5/7275; A61B 5/6803; A61B 5/1116; A61B 5/02055; A61B 2560/0242; A61B 5/7465; A61B 5/0048; A61B 2503/04; A61B 5/14542

(57) ABSTRACT

In certain embodiments, a headpiece 101 may contain the functional components described above integrated therein, and the headpiece may be releasably matable, The present invention relates to systems and methods for patient monitoring and intervention to prevent sudden unexpected death, as may occur in patients with epilepsy (SUDEP) or in infants as part of SUID, SIDS and/or suffocation. More specifically, the present invention provides a wearable device configured for monitoring a wearer and/or his environment, identifying and/or assessing death risk to the wearer, initiating communications to a caregiver that might provide an intervention or other treatment, and/or itself performing an action acting as an intervention to prevent death of the wearer. The wearable device includes particular sensors for gathering data from the wearer and/or the wearer's environment. Optionally, the wearable device may further include stimulators for delivering a death-preventing intervention stimulus to the wearer.

### CROSS-REFERENCE TO RELATED APPLICATION

This application claims the benefit of priority, under 35 U.S.C. § 119(e), of U.S. Provisional Patent Application No. 62/927,297, filed Oct. 29, 2019, the entire disclosure of which is hereby incorporated herein by reference.

### FIELD OF THE INVENTION

The present invention relates generally to systems and method for patient monitoring and/or intervention to prevent sudden unexpected death, as may occur in patients with epilepsy (SUDEP) or in infants as part of sudden unexplained infant death (SUID) and/or sudden infant death syndrome (SIDS).

### DISCUSSION OF RELATED ART

Persons may experience sudden death for various reasons. Sudden unexpected death in epilepsy (SUDEP) sudden unexplained infant death (SUID), sudden infant death syndrome (SIDS), accidental suffocation and/or “strangulation in bed” are just a few examples of types of sudden death occurrences. In such cases, persons often inadvertently smother themselves, and die from asphyxiation.

It is generally held that many SUDEP, SUID, SIDS and/or suffocation-related unexpected deaths are preventable by intervention. For example, human caregivers in proximity to the patient at the critical time might be able to provide death-prevention intervention in the nature of waking the person or helping the person to change sleeping position, e.g., to avoid a prone (face-down) sleeping position. However, caregivers may not be in proximity to a patient at all critical times, and may not be aware of a critical point in time at which as life-saving intervention is needed, even if present.

What is needed is a wearable device for monitoring the patient and/or providing death-preventing intervention when needed, even when a human caregiver is not present or in proximity to the patient.

### SUMMARY

The present invention relates to systems and methods for patient monitoring and intervention to prevent sudden unexpected death, as may occur in patients with epilepsy (SUDEP) or in infants as part of SUID, SIDS, accidental suffocation and/or “strangulation in bed.” More specifically, the present invention provides a wearable device configured for monitoring a wearer and/or the wearer's environment, identifying and/or assessing death risk to the wearer, initiating communications to a caregiver that might provide an intervention or other treatment, and/or itself performing an action acting as an intervention to prevent death of the wearer. The wearable device includes particular sensors for gathering data from the wearer and/or the wearer's environment. Optionally, the wearable device may further include stimulators for delivering a death-preventing intervention or stimulus to the wearer.

### BRIEF DESCRIPTION OF THE FIGURES

An understanding of the following description will be facilitated by reference to the attached drawings, in which:

FIG. 1 is a system diagram showing an exemplary network environment in which the present invention may be employed;

FIG. 2 is a schematic diagram of an exemplary special-purpose Monitoring and Messaging System computing device in accordance with an exemplary embodiment of the present invention;

FIG. 3 is a diagram of an exemplary risk detection device in accordance with an exemplary embodiment of the present invention;

FIG. 4 is a diagram of an exemplary risk detection device in accordance with an alternative exemplary embodiment of the present invention;

FIG. 5 is a diagram of an exemplary risk detection and intervention device in accordance with an alternative exemplary embodiment of the present invention; and

FIG. 6 is a diagram of an exemplary risk detection and intervention device in accordance with an alternative exemplary embodiment of the present invention.

### DETAILED DESCRIPTION

The present invention provides a wearable device configured for monitoring a wearer/person and/or his environment, identifying and/or assessing death risk to the wearer, initiating communications to a caregiver that might provide an intervention or other treatment, and/or itself performing an action acting as an intervention to prevent death of the wearer.

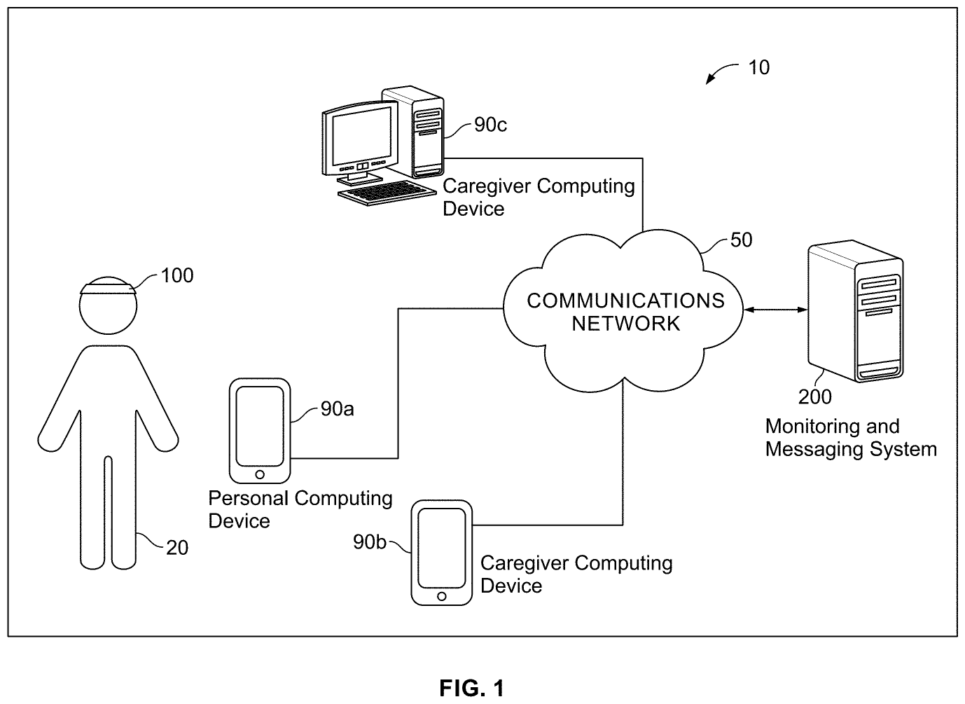

Exemplary embodiments of the present invention are discussed below for illustrative purposes. FIG. 1 is a system diagram showing an exemplary network environment in which the present invention may be employed. As shown in FIG. 1, the exemplary network environment 10 includes conventional computing hardware and software for communicating via a communications network 50, such as the Internet, etc., at the Person's Computing Device 90a (e.g., a personal computer/PC, tablet computer, smartphone or virtual assistant device, such as an Amazon Echo, Dot or other Alexa-based device commercially available from Amazon.com Inc. of Seattle Wash. and/or a comparable device such as the Google Home Mini commercially available from Alphabet Inc. of Mountain View, Calif.), and the Caregiver Computing Devices 90b, 90c (e.g., a personal computer/PC, tablet computer, smartphone or virtual assistant device, such as an Amazon Echo, Dot or Google Home Mini).

The exemplary system also includes a Monitoring and Messaging System (MMS) 200. The MMS 200 is operatively connected to the Person's and Caregiver Computing Devices 90a, 90b, 90c via the communications network 50. These systems may be existing or otherwise generally conventional systems, at least in part, including conventional software and web server or other hardware and software for communicating via the communications network 50. Consistent with the present invention, these systems may be configured, in conventional fashion, to communicate/transfer data via the communications network 50 in accordance with and for the purposes of the present invention, as discussed in greater detail below.

Further, in accordance with the present invention, the network environment 10 includes a wearable risk detection device 100 in accordance with the present invention. The risk detection device 100 is specially configured to be worn on the head of the patient/person 20 to be monitored. The risk detection device 100 includes various sensors, of various types, some of which may be arranged in selected locations on the headpiece to register with the wearer's anatomy and/or to sense conditions of the wearer and/or the wearer's environment and generate associated data. Further, the risk detection device 100 is configured to communicate data, such as gathered data, and/or other data derived from the gathered data, to the Person's Computing Device 90a, the Caregiver Computing Device 90b, 90c, and/or the MMS 200. The data communication may be performed in any suitable fashion. In one embodiment, data is communicated via short-range wireless transmission, e.g., via Bluetooth, to a nearby Patient Computing Device 90a, which may in turn communicate with the MMS 200 and/or the Caregiver Computing Device 90b, 90c. Hardware and software for enabling communication of data by such devices via such communications networks are well known in the art and beyond the scope of the present invention, and thus are not discussed in detail herein.

Gathered data may be processed at the risk detection device 100, or may be transmitted via a network for processing at a location other than the risk detection device 100, such as at the MMS 200. The data is processed to determine whether a risk state exists. If so, the risk detection device 100 or the MMS 200 may transmit data via a network to provide an alert to a caregiver, so the caregiver can provide a death-preventing intervention. Alternatively or additionally, the risk detection device 100 may further include stimulators for providing a death-preventing intervention, and the risk detection device 100 or the MMS 200 may resultingly cause the risk detection device to itself deliver a death-preventing intervention.

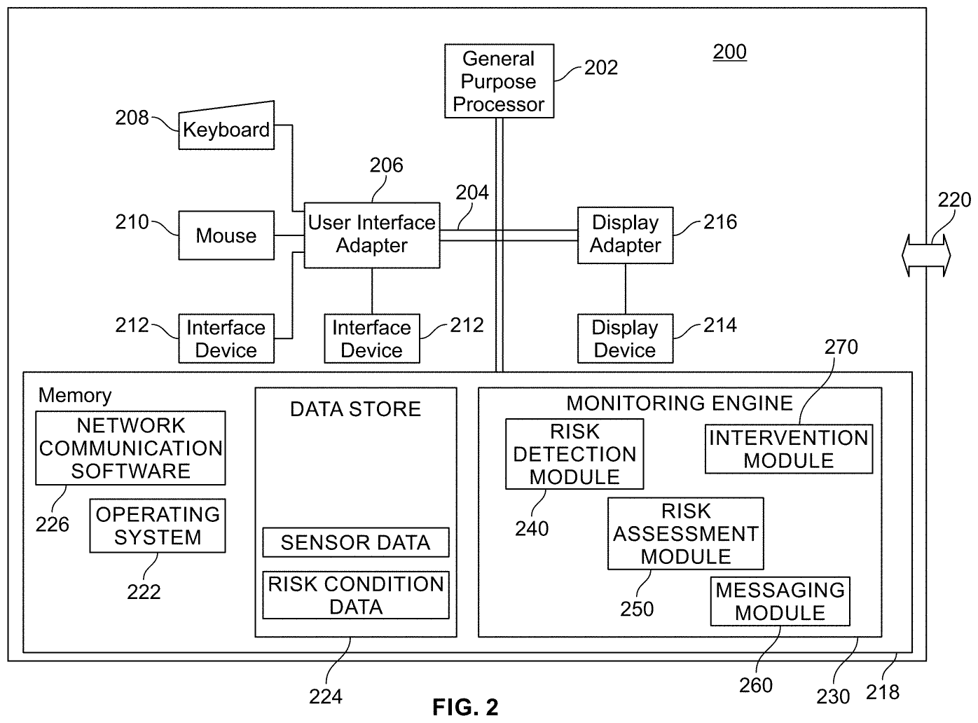

FIG. 2 is a block diagram showing an exemplary Monitoring and Messaging System (MMS) 200 in accordance with an exemplary embodiment of the present invention. The MMS 200 includes conventional computing hardware storing and executing conventional software enabling operation of a general-purpose computing system, such as operating system software 222, network communications software 226. By way of example, the communications software 226 may include conventional web server software, and the operating system software 222 may include iOS, Android, Windows, Linux software. Additionally, the MMS 200 includes specially-configured computer software stored in its memory and executable to carrying out at least one method in accordance with the present invention

Accordingly, the exemplary MMS 200 of FIG. 2 includes a general-purpose processor, such as a microprocessor (CPU), 202 and a bus 204 employed to connect and enable communication between the processor 202 and the components of the presentation system in accordance with known techniques. The exemplary presentation system 200 includes a user interface adapter 206, which connects the processor 202 via the bus 204 to one or more interface devices, such as a keyboard 208, mouse 210, and/or other interface devices 212, which can be any user interface device, such as a touch sensitive screen, digitized entry pad, etc. The bus 204 also connects a display device 214, such as an LCD screen or monitor, to the processor 202 via a display adapter 216. The bus 204 also connects the processor 202 to memory 218, which can include a hard drive, diskette drive, tape drive, etc.

The MMS 200 may communicate with other computers or networks of computers, for example via a communications channel, network card or modem 220. The MMS 200 may be associated with such other computers in a local area network (LAN) or a wide area network (WAN), and may operate as a server in a client/server arrangement with another computer, etc. Such configurations, as well as the appropriate communications hardware and software, are known in the art.

The MMS 200 is a special-purpose machine, in accordance with the present invention. Accordingly, as shown in FIG. 2, the MMS 200 includes computer-readable, processor-executable instructions stored in the memory 218 for carrying out the methods described herein. Further, the memory 218 stores certain data, e.g. in one or more databases or other data stores 224 shown logically in FIG. 2 for illustrative purposes, without regard to any particular embodiment in one or more hardware or software components.

Further, as will be noted from FIG. 2, the MMS 200 includes, in accordance with the present invention, a Monitoring and Messaging Engine (MME) 230, shown schematically as stored in the memory 218, which includes a number of modules providing functionality in accordance with the present invention, as discussed in greater detail below. These modules may be implemented primarily by specially-configured software including microprocessor-executable instructions stored in the memory 218 of the MMS 200. Optionally, other software may be stored in the memory 218 and and/or other data may be stored in the data store 224 or memory 218.

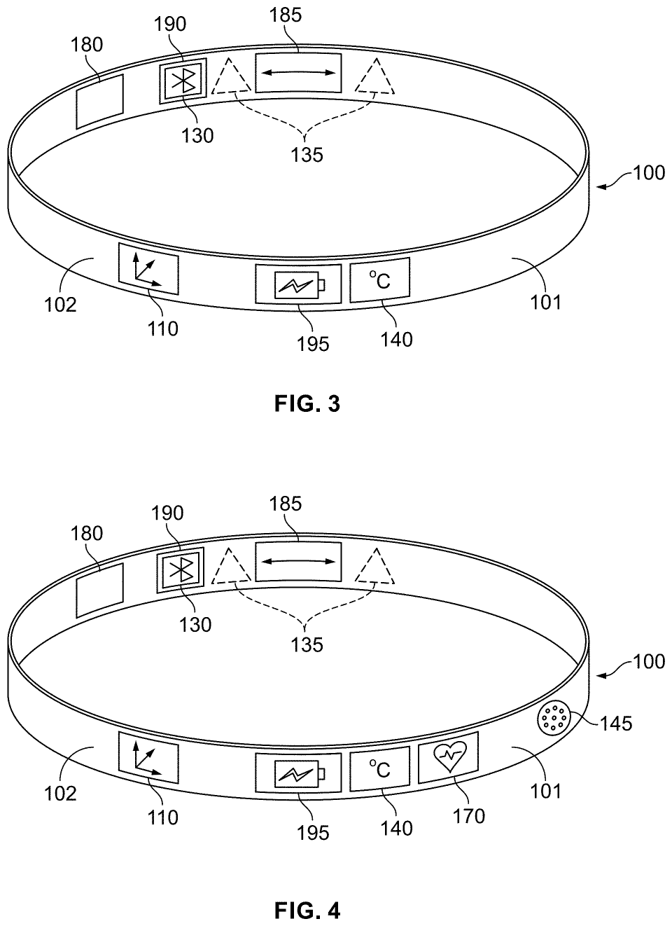

FIG. 3 is a diagram of an exemplary risk detection device 100 in accordance with an exemplary embodiment of the present invention. In this embodiment, the wearable device gathers risk detection data, but does not include an intervention stimulus delivery system. More particularly, in this exemplary embodiment, the device 100 is provided in the form of a wearable headpiece 101, such as a lightweight headband or elastic fabric or inelastic material, or a “blackout” eye mask, that can be worn encircling the head or over the forehead. In certain embodiments, the headpiece 101 may be provided as part of a beanie, cap or hat, such as a baseball cap, with the operative components described integrated therein. Alternatively, rather than a headband for encircling the head of the wearer (as shown in FIG. 3), the headpiece may be provided as a small adhesive-mountable module, medical grade tape, an eye mask, or in any other suitable form.

Notably, the device 100 includes at least one, and preferably an array, of multi-axis accelerometers and/or positional (e.g., gyroscopes) spatial orientation (collectively “positional”) sensors 110 arranged to provide an indication of the headpiece (and thus head) position. In this embodiment, the headpiece 101 includes a controller 180 operatively connected to the headpiece's positional sensors and/or other components for gathering, storing, communicating and/or processing data gathered from the sensors of the headpiece 101.

In this embodiment, the headpiece 101 further includes a temperature sensor 140, such as a thermocouple or thermometer, for measuring the person's body temperature. For example, the temperature sensor 140 may be located on the forehead portion 102 of the headpiece 101, e.g. on its inner surface, in a position to abut the forehead of a wearer of the headpiece 101. The temperature sensor 140 gathers data, e.g., temperature data, that may be used by the controller 180 to detect the presence of a predefined risk state. For example, the temperature sensor may measure a temperature indicative of a fever, which indicates an increased risk for respiratory failure.

Optionally, the controller 180 may be configured for processing data gathered from on-board sensors, and optionally, other sensors, to identify or assess risk, and optionally to initiate communication of data via a network to provide a suitable informational message to a caregiver to initiate a death-prevention intervention for the patient, or otherwise to trigger an automated intervention, as a function of data gathered from the sensors. Alternatively, processing of the data may be performed at the MMS 200 and/or the Caregiver Computing Device 90b, 90c.

Optionally, the headpiece 101 may further include a communications unit 190 for communicating data to another device, e.g., in a wired or wireless fashion, e.g., to the person's Computing Device, and/or via the network to the MMS 200 or the Caregiver Computing Device 90b, 90c. More particularly, this exemplary embodiment of the device 100 includes a wireless connection module 130 for communicating to a computer, smartphone or other computing device, and is configured to send sensor data to the MMS 200 for processing to (a) assess risk and/or (b) to send a message to provide an alert to a caregiver that may provide an intervention. In other embodiments, processing described below as performed by the MMS 200 may instead be performed at another Computing Device, or at the risk detection device 100 (e.g., via the controller 180).

Further, the headpiece 101 includes a battery 195 providing a power source for operation of the controller 180, communications unit 190, sensors, etc.

By way of example, this relatively simple embodiment of the device can detect and/or assess risk of sudden death by detecting if a person's head is in a face-down orientation (based on the sensor data gathered from the headpiece 101 worn on the head), such that asphyxiation is more likely. This may be done by comparing risk condition data (which may be default or other stored data) with sensor data (e.g., head orientation data as reflected by the positional sensor 110 and/or body temperature data as reflected by the temperature sensor 140). This may involve a simple comparison of gathered data to predetermined thresholds and/or a more complex analysis based on a predetermined risk assessment model, which may involve calculations based on data gathered from one or more sensors and/or logic-based determinations. For example, the risk condition data may reflect a certain orientation of the headpiece 101 that is associated with a head-down bodily position, and a controller 180 on the headpiece may compare current sensor data with risk condition data to determine whether a risk state condition exists that warrants an intervention.

In certain embodiments, this comparison and/or risk state determination is performed at the risk detection device 100, or at the Patient's Device 90a. In the exemplary embodiment of FIG. 2, the MMS 200 is configured to receive and store sensor data from the headpiece 101 in its data store 224, and the MMS 200 includes a Monitoring and Messaging Engine (MME) 230 that includes a Risk Detection Module (RDM) 240 that performs the above-described comparison/risk state determination at the MMS 200, to determine whether a predefined risk state condition exists.

If it is determined that a risk state condition warranting an intervention exists, the RDM 240 works in concert with a Messaging Module 260 of the MME 230 to cause the MMS 200 to send data via the network 50 to provide an alert at the Caregiver's Device 90b, 90c, in this embodiment. The caregiver may then act to provide a life-saving intervention, e.g., by rolling the patient over to avoid asphyxiation.

Further, in this exemplary embodiment, the risk detection device 100 further includes a mode sensor 185 for detecting whether the headpiece 101 is currently being worn on the head of a wearer. This may be achieved in various ways. In one embodiment, the mode sensor 185 includes a stretch sensor for determining whether the headpiece 101 is in a stretched state (as it would be when worn on the head, thereby indicating that the headpiece is being worn, and in a worn mode) or in an unstretched state (as it would be when it is not being worn on the head, thereby indicated that the headpiece is not being worn, and in an unworn mode). In such an embodiment, the controller 180 may receive state information from the mode sensor 185 and, for example, avoid sending communications to other devices that indicate a need for an intervention if the mode sensor 185 is indicating that the headpiece is not being worn at a time that the other sensors are gathering data indicative of a risk state, as the unworn state may generate sensor data falsely indicating that a risk state is present. Alternatively, if the mode sensor 185 indicates that the headpiece is not being worn at a time at which it is expected to be worn, e.g., when the person is sleeping, then the controller 180 may receive state information from the mode sensor 185 and, for example, send or cause to be sent an informational message to a caregiver that can take action to reposition the headpiece 101 on the head of the person.

In certain embodiments, the headpiece 101 may include one or more reflective fields 135 positioned on an outer surface of the headpiece 101, e.g., near the rear portion of the headpiece 101, opposite any face shield, or microphone (see below), so that the reflective fields are positioned at the back of the head when the headpiece is worn properly. These reflective fields 135 are useful for video-based monitoring of the patient's body, as they may be relatively easily observed in a video display of the patient when the patient is in a face-down position and the back of the head is exposed. This can facilitate video-based confirmation of problematic and non-problematic head positions.

FIG. 4 is a diagram of an alternative exemplary risk detection device 100 in accordance with an alternative exemplary embodiment of the present invention. In this exemplary embodiment, the risk detection device 100 is generally similar to that of FIG. 3 in structure and operation, but more complex, as it includes additional sensors permitting detection of a risk state and/or assessment/quantification of risk. Additionally, it includes a stimulus device for providing a death-preventing intervention.

Referring now to FIG. 4, this exemplary device 100 includes the same components/sensors as that of FIG. 3, and further includes a heart rate measurement sensor 170 for measuring and/or recording the person's heart rate. For example, the heart rate measurement sensor 170 may have light emitting and gathering sensors on the forehead portion 102 of the headpiece 101 (e.g., on its inner surface) in positions to abut the forehead of the wearer of the headpiece 101. The light emitting and gathering sensors 170 gathers data that may be used by the controller 180 to detect the presence of a predefined risk state, e.g., using plyethsmography techniques. For example, the light emitting and gathering sensors 170 may capture data usable to determine pre-ictal/ictal/post-ictal changes in heart rate, as well as potential arrhythmias/pauses related to a seizure occurrence.

This exemplary headpiece 101 of FIG. 4 further includes an intervention delivery system in the form of an alarm system including an audio signal-producing device 145 that may be used to awaken the person. By way of example, the alarm system may include one or more loudspeakers or other audio-producing device positioned on the headpiece 101. In such an embodiment, the controller 180 is configured to activate the alarm system to provide an audible alarm signal in response to detection of a risk state and/or assessment quantification of a risk as being sufficiently high to warrant an intervention. As described above, the risk assessment may be performed at the headpiece 101, the Person's Computing Device 90a, the Caregiver Computing Device 90b, 90c, or at the MMS. Accordingly, for example, the headpiece 101 may generate an audio signal to provide an intervention at the headpiece 101 as the result of a risk assessment performed at the MMS 200, using data gathered by sensors of the headpiece 101.

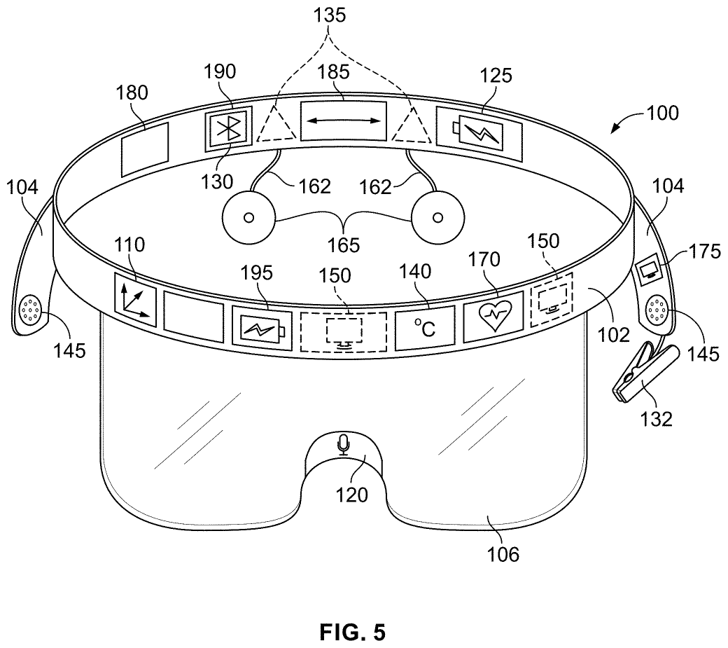

FIG. 5 is a diagram of an exemplary risk detection device 100 in accordance with an alternative exemplary embodiment of the present invention that includes an intervention delivery system. In this exemplary embodiment, the device is generally similar to that of FIG. 4 in structure and operation, but more complex, as it includes additional sensors permitting detection of a risk state and assessment/quantification of risk, and also intervention delivery systems.

In this exemplary embodiment, the risk detection device 100 is provided in the form of a wearable headpiece 101 including at least one optional earpiece 104. While this device may still be relatively thin and lightweight, this headpiece style is configured to span a greater portion of the wearer's head than the exemplary embodiments of FIGS. 3 and 4. In particular, this exemplary headpiece 101 includes an earpiece portion 104 reaching behind the wearer's ear to an earlobe, to provide an additional sensor location for reasons discussed below. Further, this exemplary headpiece 101 includes a face mask portion 106 extending downwardly from the headpiece 101, toward the wearer's nose, to provide additional structure to be used as a sensor location for reasons discussed below. Further still, the exemplary headpiece 101 includes electrodes 150 on the headpiece usable as additional sensors, and electrodes 165 attachable to the patient's neck for providing an intervention, for reasons discussed below. That said, the device 100 may be in any other suitable form and configuration.

Similar to the risk detection device of FIGS. 3-4, the exemplary headpiece 101 of FIG. 5 includes at least one, and preferably an array, of multi-axis accelerometers and/or positional sensors 110 (e.g., gyroscopes) arranged to provide an indication of head position. Similarly, the headpiece 101 includes a controller 180 for gathering and storing data gathered from the on-board sensors, which in this example is physically positioned on the rear portion of the headpiece 101. Further, the headpiece 101 similarly includes a communications unit 190 for communicating sensor and/or other data to another device, e.g., in a wired or wireless fashion, e.g., directly to the person's Computing Device 90a, and/or via the network to the MMS 200 and/or the Caregiver Computing Device 90b, 90c. Accordingly, the headpiece 101 may be used similarly to the device of FIG. 3, to provide functionality similar to that of FIG. 3, e.g. to detect a risk state as determined at the headpiece 101 and/or at the person's Computing Device 90a, and/or at the Caregiver Computing Device 90b, 90c, and/or at the MMS 200. For example, the headpiece may gather data from the position/acceleration sensor(s) 110 that is used to determine whether the person is in a prone or a supine position, which provides an indication of suffocation and SUDEP/SIDS risk. As described above, identification of a risk state may result in message to a human caregiver that may provide a life-saving intervention, e.g., rolling the patient over or waking the patient.

This exemplary headpiece 101 of FIG. 5 further includes additional sensors that may be used to detect a risk state. For example, this headpiece 101 includes a microphone 120 positioned near the nose of the wearer of the device, for listening to breath sounds and measuring respiratory rate. The microphone 120 captures an audio signal of the patient's breathing and gathers data that may be used by the controller 180 to detect the presence of a predefined risk state, such as a change in an expected breathing pattern. In one exemplary embodiment, the microphone 120 is positioned on the face mask 106, e.g., on the nasal portion of the mask.

By way of additional example, this exemplary headpiece 101 further includes a pulse oximetry sensor 132 for measuring pulse oximetry. In certain embodiments, the sensor may be configured for measuring pulse oximetry while its clip is in direct contact with the skin. In such an embodiment, for example, the sensor 132 may include a clip-like structure located on an earpiece 104 of the headpiece 101 in a position to be adjacent to or register with an ear or earlobe or the wearer of the headpiece, as these portions of the anatomy are well-suited for use to obtain pulse oximetry data. In other embodiments, the sensor may not require a clip in contact with the skin and may use, for example, a sensor positioned to lay flat against the forehead of the wearer. In such an embodiment, the sensor 132 may simply be integrated elsewhere into the headpiece 101. Any suitable sensor for measuring pulse oximetry may be used, as will be appreciated by those skilled in the art. The sensor 132 gathers data that may be used by the controller 180 (or other component) to detect the presence of a predefined risk state.

By way of additional example, this exemplary headpiece 101 further includes surface capacitive electrodes 150, e.g., on the forehead portion 102, for gathering sensor data relating to electrical activity at or measured through the skin of the forehead or the wearer.

As will be appreciated by those skilled in the art, these electrodes 150 gather electrical activity data that can be used for various purposes. In one embodiment, these electrodes 150 are used to obtain a surface electromyogram (EMG) based on electrical activity measured on the head via the electrodes 150. The electrodes 150 gather data that may be used by the controller 180 to detect the presence of a predefined risk state. For example, surface EMG may be used to determine hypoxia risk ictal/post ictal. For example, a surface EMG may reveal a pattern that may be used to determine the occurrence of tonic-clonic, clonic, or tonic seizures. Another example is that the EMG may reveal a pattern which shows the absence of effort to correct an abnormal head position or other risk state. Determining these risk states may involve the controller 180 (or another component) using filters to perform data/signal analysis using data gathered via the electrodes 150.

By way of additional example, these electrodes 150 may be used to obtain an electroencephalogram (EEG), for recording the person's electroencephalograph based on electrical activity measured on the head via the electrodes 150. The electrodes 150 gather data that may be used by the controller 180 to detect the presence of a predefined risk state. For example, the EEG data may be usable to identify a seizure occurrence. Determining these risk states may involve the controller 180 (or another component) using filters to perform data/signal analysis using data gathered via the electrodes 150.

By way of additional example, one of these electrodes 150 may be used in conjunction with another electrode, such as electrode 175 located relatively remotely from the electrode 150, for reasons that will be appreciated by those skilled in the art. These electrodes 150, 175, may be used to obtain an electrocardiogram (EKG) for measuring and/or recording the person's electrocardiogram. For example, the remote electrode 175 may be positioned remotely from electrode 150 on the ear portion 106 of the headpiece 101 in a position to abut the head and/or neck (e.g., near the mastoid bone) of the wearer of the headpiece 101. The electrodes 150, 175 gather data that may be used by the controller 180 (or another component) to detect the presence of a predefined risk state. For example, the EKG may capture data usable to determine pre-ictal/ictal/post-ictal changes in heart rate, as well as potential arrhythmias/pauses related to a seizure occurrence.

Any of these sensors may be used to detect whether a risk condition exists that may warrant an intervention. In a preferred embodiment, data from one or more of these sensors are used to assess risk level. Accordingly, in some embodiments, rather than merely detect presence or absence of a defined risk state, e.g., as performed by the controller and/or the risk detection module, an algorithmic model may process data gathered by one or more sensors to quantify or otherwise assess a risk level, e.g., by considering data gathered from more than one sensor in concert. By way of example, this risk assessment may be performed at the controller 180 of the headpiece 101, or data may be transmitted from the headpiece via the communications module 190, and the assessment may be performed by a risk assessment module at the Person's Computing Device 90a, the Caregiver Computing Device 90b, 90c, and/or the MMS 200. In the example of FIG. 2, the MMS 200 includes a Risk Assessment Module for performing the risk assessment, and then working in concert with the messaging module 260 to send data to the Caregiver Computing Device 90b, 90c or elsewhere, as desired. By way of example, the Risk Assessment module may quantify a risk level by developing a composite risk score as a function of the gathered data from one or more sensors.

Alternatively, the controller 180 may act in concert with the communication module 190 to communicate gathered data to the Person's Computing Device, Caregiver Computing Device 90b, 90c, and/or MMS 200. For example, data sent to the MMS 200 may be stored as sensor data in the data store 224 of the MMS and/or be compared to risk condition data by the Risk Detection Module 240, and to trigger messaging accordingly via the Messaging Module 260. Optionally, the controller 180 may allow for calibration and/or recalibration, e.g., after placing the headpiece 101 on the head of the wearer.

In addition to sensors permitting detection of a risk state and/or assessment/quantification of risk, the exemplary headpiece 101 of FIG. 5 further includes an intervention delivery system for providing a death-preventing intervention in an automated fashion, e.g., without the need for presence or involvement of a human caregiver.

This exemplary headpiece 101 further includes an intervention delivery system in the form of an electric stimulation system including an electric stimulation device that may be used to provide an electrical stimulus to the person. By way of example, the electric stimulation system may include a power source 125 and one or more electrodes 165 supported on elongated leads 162 extending from the headpiece 101, so that they may be positioned on the wearer's skin adjacent the next muscles of the wearer of the headpiece. Accordingly, an electrical signal may be provided directly to neck muscles to directly stimulate them and cause the neck muscles to lift the head from a face-down prone position.

By way of alternative example, the electric stimulation system may include a power source and one or more electrodes positioned along any portion of the headpiece 101, to deliver a noxious stimulus in the nature of an electric shock to awaken the person. In such an embodiment, the controller 180 may be configured to activate the electric stimulation system to provide an electric stimulus signal in response to detection of a risk state and/or assessment quantification of a risk as being sufficiently high to warrant an intervention. As described above, the risk assessment may be performed at the headpiece 101, the Person's Computing Device 90a, the Caregiver Computing Device 90b, 90c, or at the MMS. Accordingly, for example, the headpiece 101 may generate an electrical stimulation signal to provide an intervention at the headpiece 101/risk detection device 100 as the result of a risk assessment performed at the MMS 200 (or alternatively, at the controller 180), using data gathered by sensors of the headpiece 101.

Further, the headpiece 101 may be configured to monitor the wearer's response to the stimulus provided, e.g., using the headpiece's accelerometer/positional sensor 110, electrodes, etc. to monitor for movement following delivery of the stimulus. The response may then be reported, in any suitable form, e.g., quantitatively or qualitatively, e.g., as determined by the Risk Assessment Module 250, by the Messaging Module 26, e.g., by transmitting data via a network to send an appropriate informational message via another computing device, such as the Caregiver Computing Device 90b, 90c.

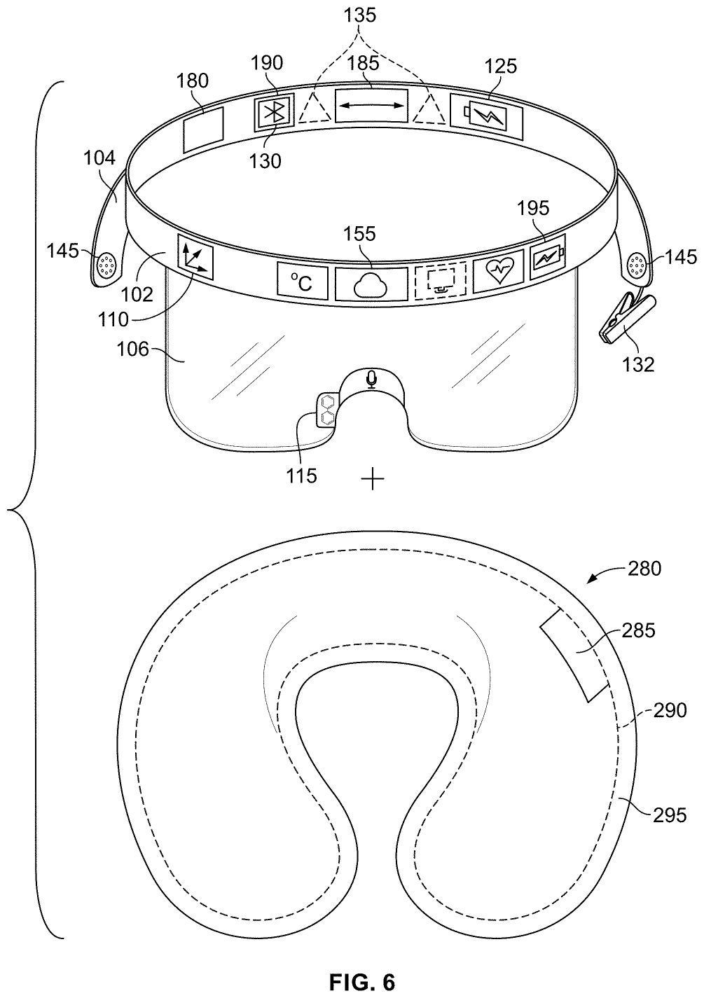

Referring now to FIG. 6, this exemplary headpiece 101 is generally similar to that of FIG. 5 in structure and operation, but further includes additional sensors and intervention delivery systems.

More particularly, the exemplary headpiece 101 of FIG. 6 further includes a smoke detector sensor 195 for detection a presence of smoke in the environment of the person. For example, the smoke detector may have a sensing portion that is located along an outer portion of the headpiece 101. The smoke detector sensor 195 gathers data that may be used by the controller 180 to detect the presence of a predefined risk state. For example, the smoke detector may gather data indicating the presence of secondary smoke. The presence of recent passive exposure to smoke may increase the risk of SUID.

Further, this exemplary headpiece 101 includes a carbon dioxide sensor 155, such as a transcutaneous carbon dioxide sensor, for detecting a carbon dioxide level in the blood. For example, the carbon dioxide sensor 155 may have a sensing portion that is located on the forehead portion 102 or face mask portion 106 of the headpiece 101. The carbon dioxide sensor 155 gathers data that may be used by the controller 180 to detect the presence of a predefined risk state. For example, the carbon dioxide sensor may gather data indicating an elevated carbon dioxide level and/or a low oxygen level that may indicate a high risk of respiratory failure.

This exemplary headpiece 101 further includes an intervention delivery system in the form of an alarm system including an audio signal-producing device 145 that may be used to awaken the person. By way of example, the alarm system may include one or more loudspeakers or other audio-producing device positioned on an earpiece 104 of the headpiece 101, adjacent the ear region of the wearer of the headpiece. In such an embodiment, the controller 180 is configured to activate the alarm system to provide an audible alarm signal in response to detection of a risk state and/or assessment quantification of a risk as being sufficiently high to warrant an intervention. As described above, the risk assessment may be performed at the headpiece 101, the Person's Computing Device 90a, the Caregiver Computing Device 90b, 90c, or at the MMS. Accordingly, for example, the headpiece 101 may generate an audio signal to provide an intervention at the headpiece 101 as the result of a risk assessment performed at the MMS 200, using data gathered by sensors of the headpiece 101.

This exemplary headpiece 101 further includes an intervention delivery system in the form of a chemical inhalant delivery system including a storage compartment 115 that may be selectively opened to release a chemical inhalant, such as ammonia-based “smelling salts,” that may be used to awaken the person. By way of example, the chemical inhalant delivery system may include a compartment 115 positioned along the forehead spanning portion 102 or on the face mask 106, near the nose of the wearer of the headpiece 101. In such an embodiment, the controller 180 is configured to activate a dispenser mechanism (such as a pump or movable shutter) of the chemical inhalant delivery system to open the compartment or otherwise release the chemical inhalant in response to detection of a risk state and/or assessment quantification of a risk as being sufficiently high to warrant an intervention.

This exemplary risk detection device 100 includes a headpiece 101 that is further configured to trigger an intervention delivery system of an external device that is not part of the headpiece 101, but rather is a physically separate and distinct external device. In this example, the external device has the form of an airbag system 280 including one or more airbags that are selectively deployable and inflatable by a gas source 285 to physically elevate the person's face out of a pillow, bedclothes, etc. to avoid asphyxiation, e.g., if a face-down prone head state is detected. By way of example, the airbag system 280 may include one or more deployable airbags 290 stored with a wearable collar 295 worn adjacent a chin region of the wearer of the headpiece. In such an embodiment, the controller 180 is configured to send a signal causing inflation of one or more of the airbags in response to detection of a risk state and/or assessment quantification of a risk as being sufficiently high to warrant an intervention. As described above, the risk assessment may be performed at headpiece 101, the Person's Computing Device 90a, the Caregiver Computing Device 90b, 90c, or at the MMS. Accordingly, for example, the headpiece 101 may initiate deployment of the airbags 290 to provide an intervention as the result of a risk assessment performed at the MMS 200, using data gathered by sensors of the headpiece 101. The intervention may be initiated by sending of a signal from an Intervention Module, such as Intervention Module 270 of MMS 200.

In an alternative embodiment, the deployable airbags 290 may be physically integrated into the headpiece 101 to eliminate the need for a separately wearable collar. For example, the headpiece 101 may be provided as part of a beanie, cap or hat, such as a baseball cap, with the components described above integrated therein, and with, for example, the deployable airbag(s) 290 integrated into the brim of the baseball cap.

In certain embodiments, a headpiece 101 may contain the functional components described above integrated therein, and the headpiece may be releasably matable, e.g. with fasteners, with a separate beanie/cap/hat or other portion that may help to support the headpiece on the head. In such an embodiment, the beanie/cap/hat portion may be made of fabric/cloth or other washable material, and the electronics/headpiece 101 may be removed to permit washing of the beanie/cap/hat portion, and then be reattached to the washed beanie/cap/hat portion prior to use by a wearer.

As discussed above, gathered data may be processed at the risk detection device 100, or remotely at the MMS 200, to determine whether a risk state exists. If so, the risk detection device 100 or the MMS 200 may transmit data via a network to provide an alert to a caregiver (so the caregiver can provide a death-preventing intervention), or to cause the risk detection device 100 itself to deliver a death-preventing intervention. The processing may be determined according to a predefined logic captures in hardware and/or software at the risk detection device and/or at the MMS, and may be configured to perform calculations and/or comparisons to predefined thresholds to determine whether risk states exist, and to trigger and alert/alarm/intervention if a risk state is found to exist. By way of example, Table 1 provides an exemplary risk table that can be used to determine whether risk states exist.

TABLE 1

| | | Low Risk | Medium Risk | High Risk |
|---|---|---|---|---|
| Primary | | | | |
| | Positional sensor | Threshold value 1 | Threshold value 2 | Threshold value 3 |
| Secondary | | | | |
| | Temperature sensor | >100.4 F. | Rise by 2+ degrees | >103 F. if not alarmed |
| | | | over 60 seconds | previously |
| | EEG seizure | | | Seizure present |
| | (electrodes) | | | |
| | EMG seizure | | | Seizure present |
| | (electrodes) | | | |
| | Heart rate sensor | | 1 SD above or below | 2 SD above or below |
| | | | the mean (over last | the mean (over last |
| | | | hour) | hour) |
| | Arrythmia (leads) | | | Any abnormal |
| | | | | rhythm |
| | Pulse oximetry sensor | | Decrease by 1 SD | <94 or 2 SD below |
| | | | | the mean, whichever |
| | | | | is greater |
| | Respiratory Rate | | 1 SD above or below | 2 SD above or below |
| | (microphone) | | the mean (over last | the mean (over last |
| | | | hour) | hour) |
| | Hypercarbia - (blood | | 2 SD above or below | 2 SD above or below |
| | CO2 sensor) | | the mean (over last | the mean (over last |
| | | | hour) | hour) |
| | Smoke (smoke sensor) | Presence of smoke | | |
| | | (last 24 hrs) | | |
| | Device off head (mode | Off head mode | | |
| | sensor) | detected | | |
| Tertiary | | | | |
| | Reactivity (positional | | | No reactivity to |
| | sensor, electrodes) | | | applied stimulus |

With reference to this exemplary table, for example, processing may be done to trigger and alert/alarm/intervention as appropriate according to the exemplary Action table set forth in Table 2 below.

TABLE 2

| Initiate Action | Primary | Secondary |
|---|---|---|
| 1 | Threshold value 3 met | None required |
| 2 | Threshold value 2 met | Any LOW RISK |
| | | event detected |
| 3 | Threshold value 1 met | Any MEDIUM RISK |
| | | event detected |
| 4 | Any Positional Sensor value | Any HIGH RISK |
| | | event detected |
| 5 | No reactivity after stimulus | None required |

Accordingly, it will be appreciated from tables above, for example, that an alert/alarm/intervention may be triggered on the basis of head position alone. For example, if the positional sensor meets/surpasses a threshold value (Threshold 3) reflective of a high-risk head position, as set forth in Initiate Action condition 1 of Table 2, then an alert/alarm/intervention is triggered in this example without regard to any other sensor values/risk conditions. By way of alternative example, an alert/alarm/intervention may be triggered on the basis of a high-risk condition alone, apart from any particular head position. For example, if any HIGH RISK event is detected (as defined in Table 1), then an alert/alarm/intervention is triggered in this example without regarding to any data from the positional sensor, as shown as Initiate Action condition 4 of Table 2. By way of additional example, an alert/alarm/intervention may be triggered on the basis of a combination of head position and other secondary conditions. For example, an alert/alarm/intervention may be triggered on the basis of a medium risk head position (Threshold 2) as reflected by the positional sensor if any low-level secondary risk is present (as indicated as Initiate Action condition 2 of Table 2), or on the basis of a low risk head position (Threshold 1) as reflected by the position sensor data if any medium-level secondary risk is present (as indicated as Initiate Action condition 3 of Table 2). By way of example, the high risk head position may correspond to a position in which the airway is impinged upon, or likely to be impinged upon, based on an average of experimental data for example, and the medium risk position and low risk position may be positions corresponding to 1 and 2 standard deviations away, respectively, from the average. It will be appreciated, for example, that any suitable algorithm may be used to determine when alerts/alarms/interventions are provided, and the corresponding type of alert/alarm/intervention to be provided. By way of example, the system may be configured to provide an alert/alarm/intervention, with respect to the conditions of Table 1, if multiple risk conditions occur simultaneously, or repeatedly, or in combination, for example.

While there have been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

What is claimed is:

1. A risk detection device for preventing sudden death, the risk detection device comprising:
a headpiece dimensioned to be worn and supported on a human head;
a power source supported on the headpiece;
a positional sensor supported on the headpiece in position to detect a position of the headpiece corresponding to a prone position of a wearer's head; and
a controller operatively connected to said power source and to said positional sensor, said controller being configured, on a recurring basis, to:
gather headpiece position data from said positional sensor;
determine whether a risk state is present as a function of position data gathered from said positional sensor; and
initiate an intervention upon determination that the risk state is present.

2. The risk detection device of claim 1, said controller is configured to determine whether the risk state is present by determining whether said position data indicates that said headpiece is in a position corresponding to a face-down position of the head of the wearer.

3. The risk detection device of claim 1, wherein said controller is configured to determine whether the risk state is present by comparing stored risk condition position data with received position data.

4. The risk detection device of claim 1, further comprising:
at least one of a temperature sensor, a heart rate measurement sensor, a transcutaneous carbon dioxide sensor, a pulse oximetry sensor, a surface capacitive electrode sensor supported on the headpiece in position to gather data from a body of a wearer of the device, the sensor being operatively connected to said power source and to said controller, said controller being configured, on a recurring basis, to:
gather data from said at least one of said temperature sensor, said heart rate measurement sensor, said transcutaneous carbon dioxide sensor, said pulse oximetry sensor, and said surface capacitive electrode sensor;
determine whether a risk state is present as a function of data gathered from said at least one of said temperature sensor, said heart rate measurement sensor, said transcutaneous carbon dioxide sensor, said pulse oximetry sensor, and said surface capacitive electrode sensor; and
initiate an intervention upon determination that the risk state is present.

5. The risk detection device of claim 4, wherein said pulse oximetry sensor comprises a clip for engaging skin of the user.

6. The risk detection device of claim 5, wherein said headpiece comprises an earpiece, and wherein said clip is supported on said earpiece in a position to be adjacent to an ear of the wearer of the headpiece.

7. The risk detection device of claim 1, further comprising a microphone supported on said headpiece near a nose of the wearer of the device, said microphone being operatively connected to said power source and to said controller, said controller being configured, on a recurring basis, to:
gather data from said microphone;
determine whether a risk state is present as a function of data gathered from said microphone; and
initiate an intervention upon determination that the risk state is present.

8. The risk detection device of claim 7, wherein said headpiece comprises a face mask portion, and wherein said microphone is supported on said face mask portion.

9. The risk detection device of claim 1, further comprising a smoke detector sensor supported on said headpiece, said smoke detector sensor being operatively connected to said power source and to said controller, said controller being configured, on a recurring basis, to:
gather data from said smoke detector sensor;
determine whether a risk state is present as a function of data gathered from said smoke detector sensor; and
initiate an intervention upon determination that the risk state is present.

10. The risk detection device of claim 1, further comprising:
a mode sensor supported on the headpiece in position to detect whether the headpiece is currently being worn on the head of the wearer, the mode sensor being operatively connected to said power source and to said controller;
said controller being configured, on a recurring basis, to:
gather state data from said mode sensor, said state data indication one of a worn state and an unworn state;
determine whether a risk state is present as a function of state data gathered from said mode sensor; and
initiate an intervention upon determination that the risk state is present.

11. The risk detection device of claim 10, wherein said mode sensor is integrated into said headpiece for determining whether said headpiece is either of a stretched state and an unstretched state.

12. The risk detection device of claim 1, further comprising a reflective field positioned on an outer surface of said headpiece.

13. The risk detection device of claim 1, further comprising:
a communications unit for communicating data via a communications network.

14. The risk detection device of claim 13, wherein said communications unit is configured to communicate data to a caregiver computing device to provide an informational message at the caregiver computing device.

15. The risk detection device of claim 13, wherein said communications unit is configured to communicate data to a caregiver computing device to provide an alert signal at the caregiver computing device.

16. The risk detection device of claim 1, wherein said headpiece is dimensioned for encircling the head of the wearer and constructed as one of a headband, an eye mask, and a hat.

17. The risk detection device of claim 1, further comprising an intervention delivery system for providing a death-preventing intervention in an automated fashion.

18. The risk detection device of claim 17, wherein said intervention delivery system comprises:
an electric stimulation system comprising:
a power source; and
at least one electrode supported on said headpiece;
said controller being configured to selectively activate said electric stimulation system to provide an electric stimulus signal to the wearer of the device in response to detection of a risk state.

19. The risk detection device of claim 17, wherein said intervention delivery system comprises:
an electric stimulation system comprising:
a power source; and
at least one electrode supported on an elongated lead extending from said headpiece;
said controller being configured to selectively activate said electric stimulation system to provide an electric stimulus signal to the wearer of the device in response to detection of a risk state.

20. The risk detection device of claim 17, wherein said intervention delivery system comprises:
an audio signal-producing device operatively coupled to said controller and said power source, said audio signal-producing device being operable to produce an audio signal for awakening the wearer of the device;
said controller being configured to selectively activate said audio signal-producing device in response to detection of a risk state.

21. The risk detection device of claim 17, wherein said intervention delivery system comprises:
a chemical inhalant delivery system operatively coupled to said controller and said power source, said chemical inhalant delivery system comprising:
a storage compartment supported on the headpiece;
a chemical inhalant stored in the storage compartment; and
a dispensing mechanism operable to release chemical inhalant from the storage compartment;
said controller being configured to selectively activate said dispensing mechanism to release chemical inhalant in response to detection of a risk state.

22. The risk detection device of claim 17, wherein said intervention delivery system comprises:
an airbag system operatively coupled to said controller and said power source, said airbag system comprising:
a gas source;
an inflatable airbag in fluid communication with said gas source to be inflated upon release of gas from said gas source;
said controller being configured to selectively release gas from said gas source in response to detection of a risk state.

23. The risk detection device of claim 22, wherein said airbag system is supported on said headpiece.

24. The risk detection device of claim 22, wherein said airbag system is distinct from said headpiece.

25. A risk detection device for preventing sudden death, the risk detection device comprising:
a headpiece dimensioned to be worn and supported on a human head;
a power source supported on the headpiece;
a sensor supported on said headpiece in position to generate data by sensing a condition of one of a wearer of the risk detection device and an ambient environment of the wearer of the risk detection device; and
a communications unit operatively connected to said power source and to said sensor, said communications unit being configured, on a recurring basis, to transmit data gathered from said sensor to a device capable of determining whether a risk state is present as a function of position data gathered from said sensor.

26. A risk detection system for preventing sudden death, the risk detection system comprising:
a risk detection device comprising:
a headpiece dimensioned to be worn and supported on a human head;
a power source supported on the headpiece;
a sensor supported on the headpiece in position to generate data by sensing a condition of one of a wearer of the risk detection device and an ambient environment of the wearer of the risk detection device; and
a communications unit operatively connected to said power source and to said sensor; and
a monitoring and messaging system comprising:
a processor;
a memory; and
processor-executable instructions stored in the memory and configured to control the monitoring and messaging system to:
receive sensor data from the risk detection device via the communications network;
determine whether a risk state is present as a function of the sensor data; and
initiate an intervention upon determination that the risk state is present.
